(19) United States
(12) Patent Application Publication
(10) Pub. No.: US 2024/0123211 A1
(43) Pub. Date: Apr. 18, 2024

(54) UNIVERSAL FLUID CONNECTOR ASSEMBLY WITH A REGULATING VALVE COMPONENT

(71) Applicant: CareFusion 303, Inc., San Diego, CA (US)

(72) Inventors: Amarsinh Deeliprao JADHAV, Bangalore (IN); Rahul MALVIYA, British Columbia (CA); Atharva SHETYE, Bangalore (IN); Anand VISWANATHAN, Chennai (IN)

(21) Appl. No.: 18/463,770

(22) Filed: Sep. 8, 2023

Related U.S. Application Data
(60) Provisional application No. 63/416,891, filed on Oct. 17, 2022.

(51) Int. Cl. A61M 39/10

(52) U.S. Cl. CPC A61M 39/10; A61M 2039/1061; A61M 2039/1077

(57) ABSTRACT

Valve components positioned in fluid connector assemblies, including universal fluid connector assemblies, are shown. A valve component is designed to open or close based on fluid pressure from a medical fluid applied to the valve component. Normally, when no force is acting on the valve component, the valve component is in an open position and portions of the valve components are spaced apart by a threshold force, thus forming a valve chamber. However, in an exemplary embodiment, when an applied force by the medical fluid is at least at the threshold force, the portions of the valve component contact each other and the valve component transitions to a closed position, thereby preventing the medical fluid from passing through the valve component, and accordingly, preventing the medical fluid from passing through the fluid connector assembly.

### CROSS REFERENCE TO RELATED APPLICATION

This application claims the benefit of priority under 35 U.S.C. § 119 to Provisional application for Patent Ser. No. 63/416,891, entitled “UNIVERSAL FLUID CONNECTOR ASSEMBLY WITH A REGULATING VALVE COMPONENT,” filed on Oct. 17, 2022, the entire contents of which is incorporated herein by reference.

### TECHNICAL FIELD

The present disclosure generally relates to a connector for connecting a medical container, such as a syringe, to a vascular access device for infusion injection of a medical fluid to a patient, and particularly to pressure-regulating valve for infusion injection of the medical fluid at safe infusion pressures.

### BACKGROUND

In general, vascular access devices are inserted into veins via peripheral or central vessels. Vascular access devices can be used for infusing fluid (e.g., saline solution, blood, medicaments, and/or total parenteral nutrition) into a patient, withdrawing fluids (e.g., blood) from a patient, and/or monitoring various parameters of the patient's vascular system.

However, vascular access devices can become occluded. To ensure vascular access devices are used properly and do not become occluded, standards of practice have been developed. These standards include a cleaning procedure, which is commonly referred to as a flush procedure. These flush procedures maintain the patency of the vascular access device.

Flush procedures may be enhanced by use of a syringe specifically designed to generate lower injection pressure, such as for instance a 10 milliliter (“mL”) diameter syringe barrel, or by use of a “push-pause” or pulsatile flushing technique to remove debris or residue in the catheter that may cause occlusion or other undesirable effects.

However, fast injection of flush fluid into peripheral intravenous (“IV”) lines leads to transient pressure build-up within the vein where the catheter is sited. This pressure may lead to vein damage (e.g., rupture or collapse) and infusate infiltration/extravasation, causing clinical complications and the need to replace the peripheral IV catheter.

Typical infusion or IV sets are constructed by joining polymeric tubing segments to polymeric components, many of which use needle-free connectors. These IV sets can be used with infusion pumps or gravity systems to provide fluids via a catheter to a user, such as a patient. Typical needle-free connectors do not provide an indication of the infusion pressure of the fluid administered through the connector. However, pressure regulation is needed to provide infusion therapy for specific conditions such as for chemotherapy, infusion in neonates, infusion in geriatric patients, power injection, etc.

For instance, the pressure applied to the blood component should not exceed 300 mm Hg (5 psi) as this may result in hemolysis or bag breakage. As another example, during power injection, the IV fluid needs to be injected in bolus without control. As yet another example, United States Department of Health guidelines indicate that the infusion pressure should not exceed 25 pounds per square inch (psi), as infusion pressures higher than 25 psi may damage blood vessels and negatively affect patient health and comfort.

The description provided in the background section should not be assumed to be prior art merely because it is mentioned in or associated with the background section. The background section may include information that describes one or more aspects of the subject technology.

### SUMMARY

In accordance with at least some embodiments disclosed herein is the realization that medical devices (e.g., catheters) often undergo complications due to drug infusions at relatively high fluid pressures, such infiltration/extravasation, occlusion/medical failure, dislodgement, vein damage, and catheter replacement.

Aspects of the present disclosure provide a fluid connector assembly with a valve component that responds to an applied force provided by a medical fluid by closing and preventing fluid flow through the fluid connector assembly. As a non-limiting example, the valve component allows medical fluid to pass therethrough so long as the force provided by the medical fluid is either at or below a threshold force, and subsequently closes when the force provided by the medical fluid is greater than the threshold force. Additionally, the fluid connector assembly may include a component designed to connect to various medical fluid delivery devices without modifications, thus allowing the fluid connector assembly to act as a universal connector.

Accordingly, aspects of the present disclosure provide a fluid connector assembly comprising a housing comprising an internal chamber that forms a fluid path for the medical fluid, and a valve component disposed in the internal chamber, the valve component comprising an inner portion, an outer portion, and a valve chamber between the outer portion and the inner portion, wherein the inner portion is movable between i) a first position, wherein the inner portion is separated from the outer portion, and ii) a second position, wherein the inner portion contacts the outer portion to seal the valve chamber and close the fluid path.

Some instances of the present disclosure provide a fluid connector assembly comprising a housing comprising a housing chamber, and a valve component disposed in the housing chamber, the valve component comprising a first portion, a second portion, and a valve chamber disposed between the first portion and the second portion, wherein responsive to receiving the medical fluid, the first portion moves relative to the second portion and closes the valve chamber.

Some instances of the present disclosure provide a method for regulating a medical fluid through a fluid connector assembly, the method comprising, at a valve component located in the fluid connector assembly: receiving, at a first portion of the valve component, the medical fluid; actuating, based on receiving the medical fluid, the first portion relative to a second portion of the valve component; and closing, when the first portion contacts the second portion, a valve chamber of the valve component, the valve chamber located between the first portion and the second portion, wherein the valve chamber, when closed, prevents the medical fluid from passing through the fluid connector assembly.

Accordingly, the present application addresses several operational challenges encountered in prior infusion applications in which high fluid pressure reduces efficiency of medical fluid delivery and causes damage to a medical device or injury to a patient.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates an IV set coupled to a patient, in accordance with aspects of the present disclosure.

FIG. 2 illustrates a perspective view of the fluid connector assembly in use with a fluid delivery device, in accordance with aspects of the present disclosure.

FIG. 3 illustrates a perspective view of the fluid connector assembly used with additional needle-free devices, in accordance with aspects of the present disclosure.

FIG. 4 illustrates a perspective view of a fluid connector assembly, in accordance with aspects of the present disclosure.

FIG. 5 illustrates an exploded view of the fluid connector assembly, in accordance with aspects of the present disclosure.

FIG. 6 illustrates a partial cross-sectional view of the fluid connector assembly, in accordance with aspects of the present disclosure.

FIG. 7 illustrates a perspective view of the valve component, in accordance with aspects of the present disclosure.

FIG. 8 illustrates a partial cross-sectional view of the valve component shown in FIG. 7, taken along line 8-8, in accordance with aspects of the present disclosure.

FIG. 9 illustrates a partial cross-sectional view of the fluid connector assembly, showing the valve component permitting fluid flow through the fluid connector assembly, in accordance with aspects of the present disclosure.

FIG. 10 illustrates a partial cross-sectional view of the fluid connector assembly, showing the valve component blocking fluid flow through the fluid connector assembly, in accordance with aspects of the present disclosure.

FIG. 11 illustrates an alternate valve component, in accordance with aspects of the present disclosure.

FIG. 12 illustrates a partial cross-sectional view of the valve component shown in FIG. 11, taken along line 12-12.

FIG. 13 illustrates an alternate valve component, in accordance with aspects of the present disclosure.

FIG. 14 illustrates a partial cross-sectional view of the valve component shown in FIG. 13, taken along line 14-14.

FIG. 15 illustrates a flowchart showing a method for regulating a medical fluid through a fluid connector assembly, in accordance with aspects of the present disclosure.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present disclosure includes various features and advantages of a universal connector with a valve component that regulates pressure by closing when the applied pressure (proportional to the applied force) to the valve component reaches or exceeds, depending on the design specification, a threshold pressure, which is a function of one or more features of the valve component. Valve components described herein may include elastic capabilities allowing the valve to open when the pressure applied by the medical fluid is at or below, depending on the design specification, the threshold pressure. Accordingly, in some exemplary embodiments, a valve component may include an elastomer material.

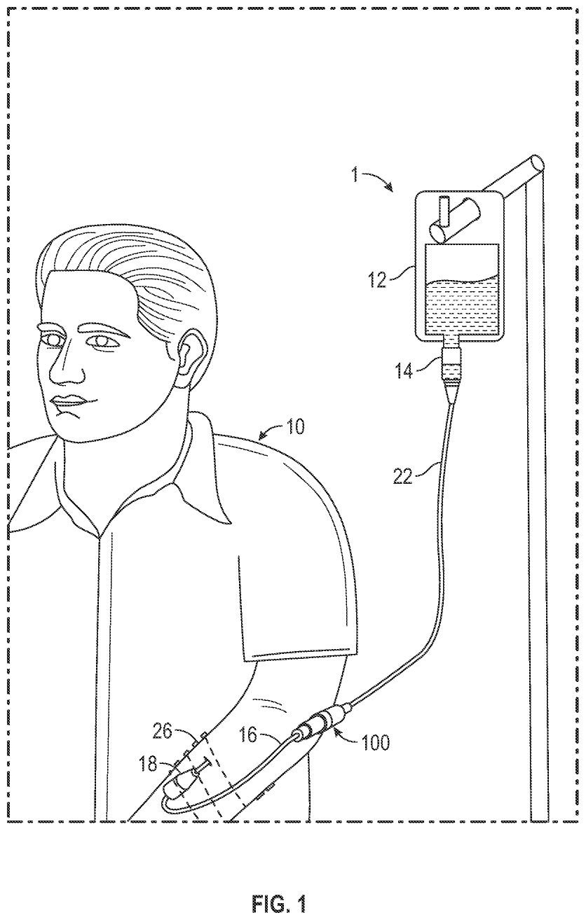

Referring now to the figures, FIG. 1 illustrates an IV set 1 coupled to a patient 10, in accordance with aspects of the present disclosure. The IV set 1 includes a medicament bag 12, a drip chamber 14, and tubing 22. The tubing 22 extends between the drip chamber 14 and a fluid connector assembly 100 of the IV set 1. To resist unintended dislodgement or disconnection of the tubing 16 or the catheter 18 from the patient, tape 26 is placed over the tubing 16 and the catheter 18, so that the tape 26 engages the tubing 16, the catheter 18, and the patient 10.

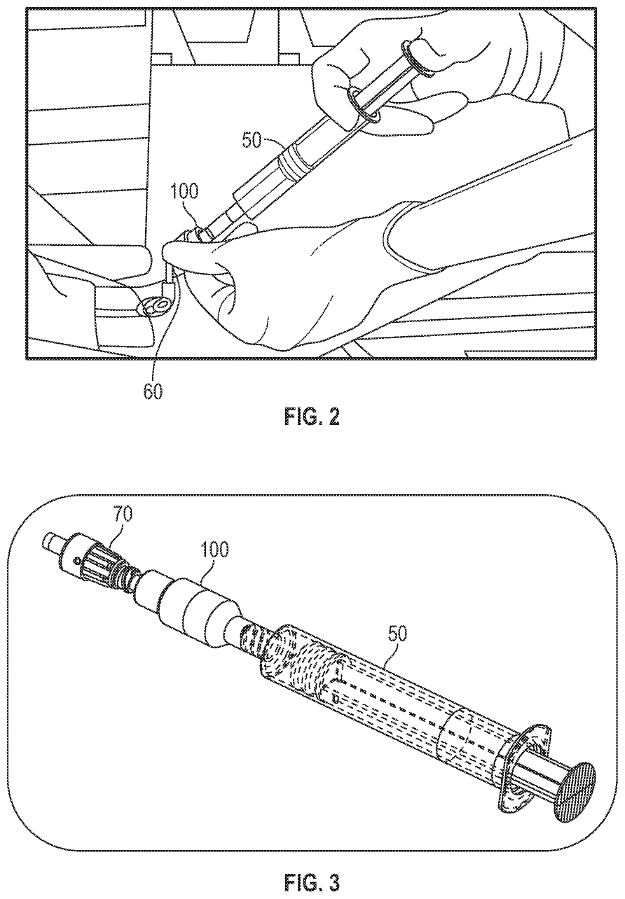

FIG. 2 illustrates the fluid connector assembly 100 in use with a fluid delivery device 50, in accordance with aspects of the present disclosure. In some embodiments, the fluid delivery device 50 includes a syringe, such as a needle-free syringe. Accordingly, in some embodiments, the fluid connector assembly 100 can take the form of a needle-free fluid connector assembly. For some exemplary IV applications, the fluid delivery device 50 may be used for rapid injections of medication, sometimes referred to as a “push” or “bolus” to quickly send a one-time dose of medication into a patient's bloodstream.

FIG. 3 illustrates a perspective view of the fluid connector assembly 100 used with additional needle-free devices, in accordance with aspects of the present disclosure. As shown, the fluid connector assembly 100 is used with the fluid delivery device 50 at a fluid inlet of the fluid connector assembly 100. Additionally, the fluid connector assembly 100 is used with a connector 70 at a fluid outlet of the fluid connector assembly 100. In some embodiments, the connector 70 includes a needle-free connector, such as a luer. The connector 70 can connect to another medical device (not shown in FIG. 3), such as a catheter line or an IV, as non-limiting examples. Alternatively, the fluid delivery device 50, the fluid connector assembly 100, and the connector 70 can collectively be used in a flush operation to clean a device (e.g., catheter) connected to the connector 70.

FIGS. 1-3 show that the fluid connector assembly 100 is suitable for use with multiple devices. As a result, the fluid connector assembly 100 may be referred to as a universal fluid connector assembly, as the fluid connector assembly 100 can be used in multiple medical applications without modification(s) required for integration.

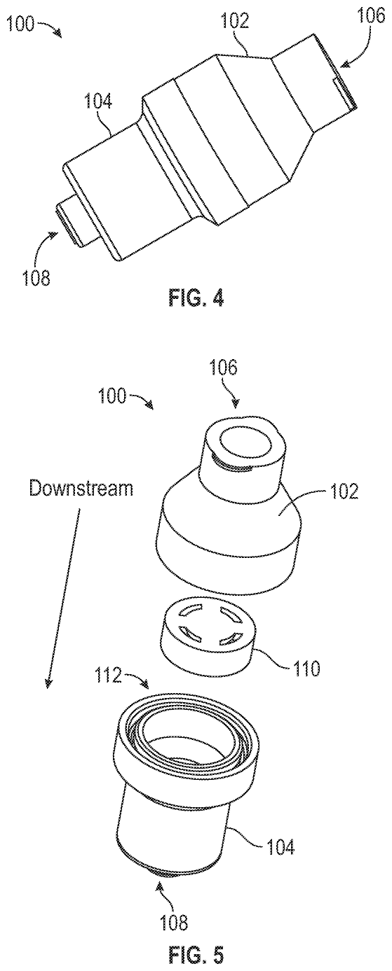

FIG. 4 illustrates a perspective view of the fluid connector assembly 100, in accordance with aspects of the present disclosure. The fluid connector assembly 100 may include a connector component 102 and a connector component 104 coupled with the connector component 102. The connector components 102 and 104 form a housing for the fluid connector assembly 100. Also, the connector components 102 and 104 may be referred to as a first connector component and a second connector component, respectively. However, “first” and “second” may be used interchangeably. The coupling between the connector components 102 and 104 may include a welding operation (e.g., ultrasonic welding), adhesives, or the like. The connector component 102 is designed to connect with various fluid delivery devices, including several needle-free devices, as non-limiting examples.

The connector component 102 includes a fluid inlet 106, and the connector component 104 includes a fluid outlet 108. The fluid inlet 106 and the fluid outlet 108 form a fluid inlet and fluid outlet, respectively, for the fluid connector assembly 100. The fluid inlet 106 and the fluid outlet 108 are cylindrical, or generally cylindrical, bodies with circular cross sections. However, other shapes are possible.

The fluid connector assembly 100 includes a fluid regulating device carried within the fluid connector assembly 100. FIG. 5 illustrates an exploded view of the fluid connector assembly 100, in accordance with aspects of the present disclosure. As shown, the fluid connector assembly 100 includes a valve component 110 designed to regulate fluid through the fluid connector assembly 100. For example, fluid passing through the fluid inlet 106 can further pass through the valve component 110, when open, and through the fluid outlet 108. Conversely, the valve component 110, when closed, can prevent or prohibit fluid passing through the fluid inlet 106 from further passing through the fluid outlet 108. The opening or closing of the valve component 110 is based on the amount of applied pressure received by the valve component 110 from a fluid (e.g., a medical fluid). This will be shown in greater detail below. When the fluid connector assembly 100 is assembled, the valve component 110 is disposed in a compartment 112 of the connector component 104. In some embodiments (not shown), the connector component 102 includes a compartment similar to the compartment 112) to carry the valve component 110.

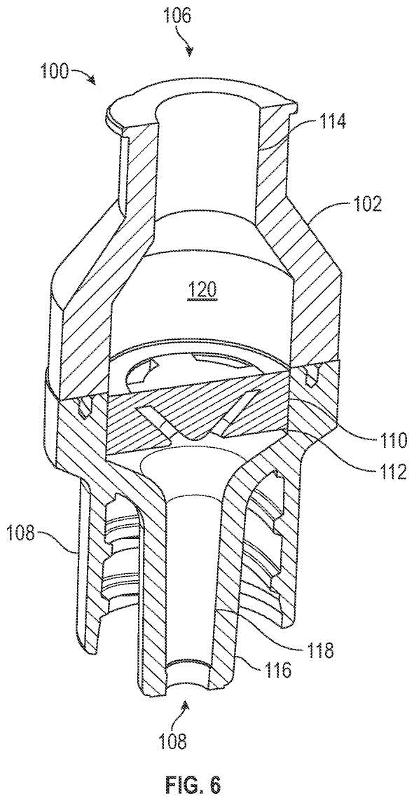

FIG. 6 illustrates a partial cross-sectional view of the fluid connector assembly 100, in accordance with aspects of the present disclosure. As shown, the connector component 102 includes a channel 114. Additionally, the connector component 104 includes a post 116 that includes a channel 118. The compartment 112 and the channels 114 and 118 can collectively form an internal chamber 120 of the fluid connector assembly 100 through which fluid passes through the fluid connector assembly 100 entering the fluid inlet 106 and exiting the fluid outlet 108, provided the valve component 110 is open. Accordingly, the internal chamber 120 forms a fluid path. As shown in FIG. 6, the valve component 110 is sealed with the connector 104 in the compartment 112. As a result, fluid can flow through, and not around, the valve component 110 when the valve component 110 is open.

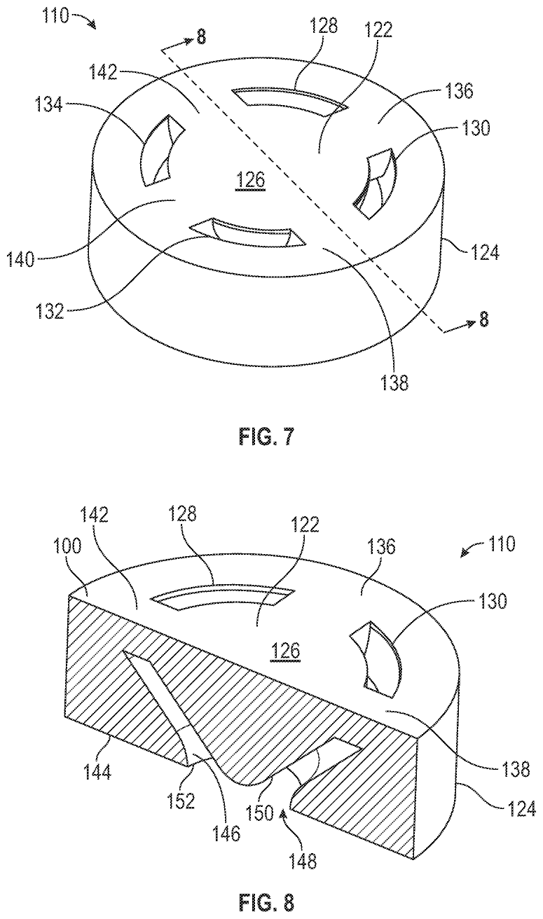

FIG. 7 illustrates a perspective view of the valve component 110, in accordance with aspects of the present disclosure. As shown, the valve component 110 includes an inner portion 122 (or inner region) and an outer portion 124 (or outer region). Based on the respective positions, the inner portion 122 is positioned within the outer portion 124. Put another way, the outer portion 124 surrounds the inner portion 122. Also, the outer portion 124 includes a cylindrical body, and as a result, the valve component 110 takes the form of a cylindrical valve component. However, other shapes are possible.

The valve component 110 further includes a surface 126 with several openings formed in the surface 126. For example, the valve component 110 includes an opening 128, an opening 130, an opening 132, and an opening 134, each of which is formed in the surface 126. The openings 128, 130, 132, and 134 each represents voids in the surface 126 through which fluid can enter the valve component 110. Accordingly, the surface 126 may be referred to as a fluid receiving surface. Additionally, the valve component 110 includes several ledges that connect the inner portion 122 with the outer portion 124. For example, the valve component 110 includes a ledge 136, a ledge 138, a ledge 140, and a ledge 142. The ledges 136, 138, 140, and 142 can collectively suspend the inner portion 122 from the outer portion 124. While a discrete number of openings (i.e., openings 128, 130, 132, and 134) and ledges (i.e., ledges 136, 138, 140, and 142) are shown, the number of openings and ledges may vary in other embodiments.

In some embodiments, the valve component 110 is formed from a flexible material, such as elastomer. As a result, the valve component 110 is flexible in at least some locations.

FIG. 8 illustrates a partial cross-sectional view of the valve component 110 shown in FIG. 7, taken along line 8-8, in accordance with aspects of the present disclosure. In addition to the surface 126, the valve component 110 further includes a surface 144. The surfaces 126 and 144 may be referred to as a first surface and as a second surface, respectively. However, “first” and “second” may be used interchangeably. The valve component 110 includes an opening 146 formed in the surface 144. The opening 146 represents voids in the surface 144 through which fluid can exit the valve component 110.

Additionally, the valve component 110 includes a valve chamber 148 that forms a void or space within the valve component 110. Additionally, the valve chamber 148 represents a separation between the inner portion 122 and the outer portion 124. Also, the valve chamber 148 is fluidly connected to the openings 128, 130, 132, and 134 (some of which are shown in FIG. 7) of the surface 126 and the opening 146 of the surface 144. Accordingly, fluid passing through the openings 128, 130, 132, and 134 may enter the valve chamber 148 and then pass through the opening 146.

As shown in FIGS. 7 and 8, the valve component 110 is in an open position. Accordingly, fluid entering any one or more of the openings 128, 130, 132, and 134 can pass through the valve chamber 148 and exit the opening 146. However, the valve component 110, in a closed position, prevents fluid passage entering any one of the openings 128, 130, 132, and 134 from exiting through the opening 146, as the valve chamber 148 collapses and closes in the closed position. In this regard, the inner portion 122 includes a surface 150 and the outer portion 124 includes a surface 152. The inner portion 122 and the outer portion 124 may each include a symmetrical body, with the respective surfaces (i.e., surfaces 150 and 152) being parallel, or at least approximately parallel, to each other. When the surface 150 is not in contact with the surface 152 (as shown in FIG. 8), the valve component 110 is in the open position. Conversely, when the inner portion 122 moves relative to the outer portion 124 and contacts the outer portion 124, the surface 150 contacts the surface 152 (and the surfaces 150 and 152 are no longer parallel), and the valve component 110 is in the closed position.

Referring again to the valve component 110 in FIG. 7, the ledges 136, 138, 140, and 142 provide separation between the inner portion 122 and the outer portion 124, with the separation generally defining the valve chamber 148. The ledges 136, 138, 140, and 142 may define a threshold pressure that maintains at least a partial separation between the inner portion 122 and the outer portion 124 such that the inner portion 122 and the outer portion 124 do not contact each other, thus maintaining the valve component 110 in the open position. The threshold pressure may be a function of one or more of the size and shape of the ledges 136, 138, 140, and 142, the material makeup (e.g., elastomer) of the ledges 136, 138, 140, and 142, the number of ledges (i.e., more or less than what is shown), and the area of the surface 126. Accordingly, design considerations of the surface 126 and/or ledges 136, 138, 140, and 142 can be considered in determining the threshold pressure. In some embodiments of the present disclosure, the inner portion 122 has a first area defined by the first surface 126, and the inner portion 122 has a second area defined by the second surface 144, where the first area is greater than the second area.

In some embodiments, the threshold pressure is 25 psi, or at least approximately 25 psi. However, the desired threshold pressure can be less than or greater than 25 psi based on the specifications of the valve component 110. In some embodiments, the valve component 110 transitions from the open position to the closed position when the fluid provides an external pressure that is at least at the threshold pressure, and the valve component 110 transitions back to the open position when the external pressure is less than the threshold pressure. Alternatively, in some embodiments, the valve component 110 transitions from the open position to the closed position when the fluid provides an external pressure that is greater than the threshold pressure, and the valve component 110 transitions back to the open position when the external pressure is at least at the threshold pressure. Also, pressure is directly proportional to force, and as a result, a comparison between the external pressure and the threshold pressure is akin to a comparison between an external force and a threshold force, respectively.

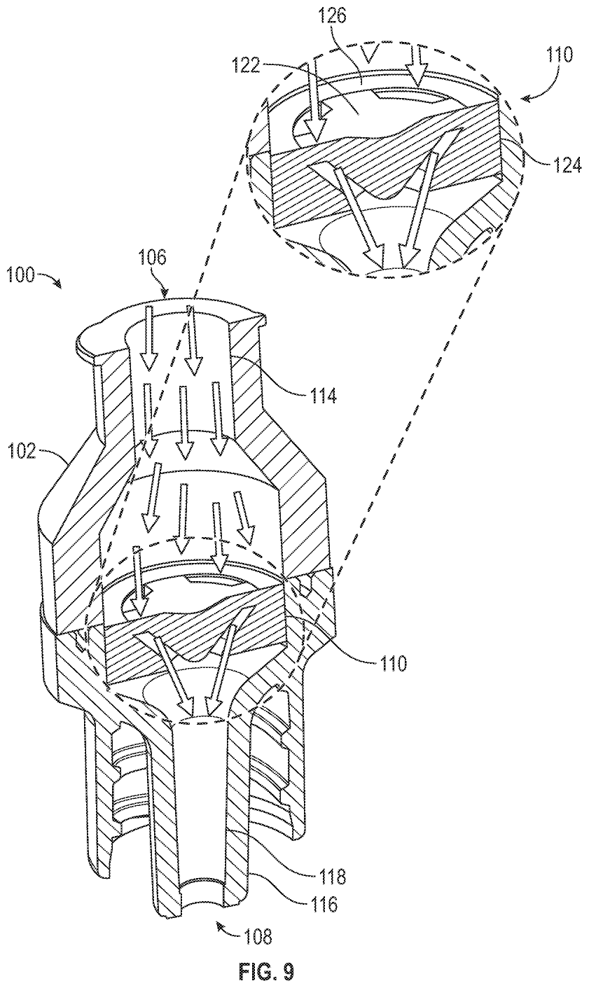

FIG. 9 illustrates a partial cross-sectional view of the fluid connector assembly 100, showing the valve component 110 permitting fluid flow through the fluid connector assembly 100, in accordance with aspects of the present disclosure. The arrows represent fluid flow in a downstream, or generally downstream, direction through the fluid connector assembly 100. When fluid enters the fluid inlet 106 of the fluid connector assembly 100, the fluid passes through the channel 114 of the connector component 102 and through the valve component 110 by way of the openings (i.e., openings 128, 130, 132, and 134, shown in FIG. 7) of the valve component 110, and subsequently through the channel 118 of the post 116, where the fluid exits the fluid outlet 108 of the fluid connector assembly 100.

As shown in the enlarged view, the valve component 110 alters at least some of its features in response to the fluid flow. For example, when the fluid provides sufficient pressure (i.e., external pressure) to the surface 126 of the valve component 110, the inner portion 122 may deform. Additionally, the inner portion 122 moves with respect to the outer portion 124. The degree to which the inner portion 122 moves is dependent upon the applied pressure provided by the fluid. The inner portion 122 and the outer portion 124 may be referred to as a movable portion and a stationary portion, respectively. The ledges (i.e., ledges 136, 138, 140, and 142) provide flexibility, and in response to the pressure provided by the fluid, will bend or flex to allow the relative movement of the inner portion 122. However, as shown in FIG. 9, the valve component 110 remains in the open position. Accordingly, the force (proportional to the pressure) provided by the fluid flowing through fluid connector assembly 100 may alter the valve component 110. However, as long as the force is below a threshold force, the valve component 110 remains in the open position.

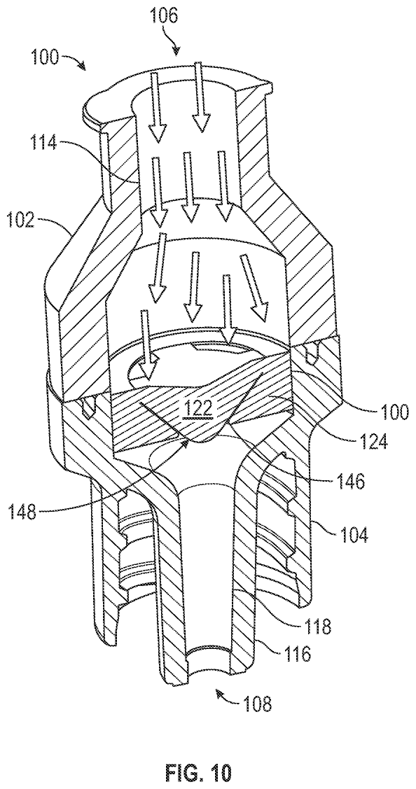

FIG. 10 illustrates a partial cross-sectional view of the fluid connector assembly 100, showing the valve component 110 blocking fluid flow through the fluid connector assembly 100, in accordance with aspects of the present disclosure. The arrows represent fluid flow in a downstream, or generally downstream, direction through the fluid connector assembly 100. When fluid enters the fluid inlet 106 of the fluid connector assembly 100, the fluid passes through the channel 114 of the connector component 102. However, as shown in FIG. 10, the valve component 110 is in the closed position, as the inner portion 122 moves, based on pressure provided by the fluid, relative to the outer portion 124 in a manner such that the inner portion 122 contacts the outer portion 124. As a result, the valve chamber 148 collapses and closes, and closes off the opening 146. Accordingly, fluid does not pass through the channel 118 of the post 116 of the connector component 104, and does not flow out of the fluid outlet 108.

FIGS. 11-14 show and describe valve components with different aspects. The valve components shown and described in FIGS. 11-14 may include several features shown and described for the valve component 110 (shown in FIG. 7, for example), and the valve components shown and described in FIGS. 11-14 may be substituted for the valve component 110.

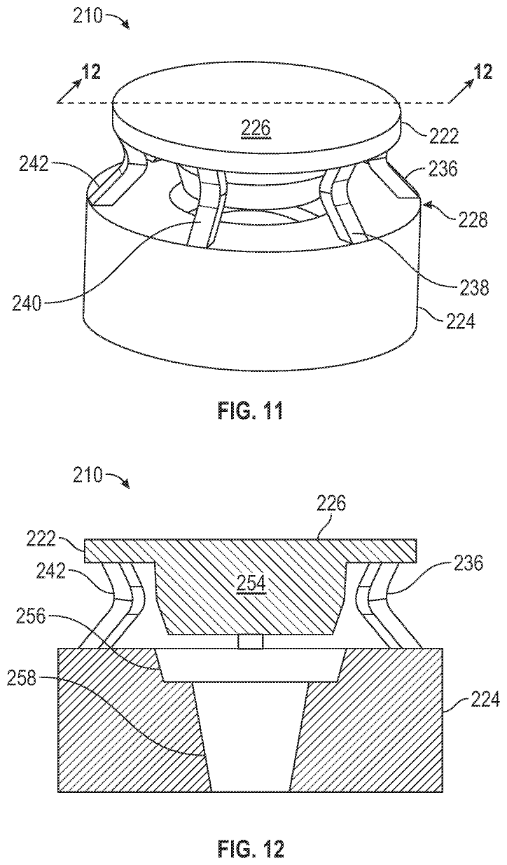

FIGS. 11 and 12 illustrate an alternate valve component 210, in accordance with aspects of the present disclosure. As shown, the valve component 210 includes an upper portion 222 and a lower portion 224. Based on the respective positions, the upper portion 222 is positioned above the lower portion 224. The upper portion 222 and the lower portion 224 may be referred to as a first portion and a second portion, respectively. However, “first” and “second” may be used interchangeably. The upper portion 222 and the lower portion 224 are separated by several ledges. For example, the valve component 210 includes a ledge 236, a ledge 238, a ledge 240, and a ledge 242. The ledges 236, 238, 240, and 242 may be representative of one or more additional ledges. Also, an opening is defined in part by adjacent ledges. For example, an opening 228 (representative of additional openings) is defined in part by ledges 236 and 238 (i.e., adjacent ledges).

FIGS. 11 and 12 show the valve component 210 is in an open position. As shown in FIG. 12, the upper portion 222 includes a protrusion 254 and the lower portion 224 includes a compartment 256 and a channel 258 fluidly connected to the compartment 256. In the open position, fluid flows between the upper portion 222 and the lower portion 224, into openings (i.e., opening 228 in FIG. 11) between adjacent ledges, through the compartment 256, and then exits the valve component 210 through the channel 258. However, the valve component 210 can transition from the open position to a closed position when a fluid applies sufficient pressure to a surface 226 of the upper portion 222. When the applied pressure by the fluid is greater than a threshold pressure provided by the ledges 236, 238, 240, and 242 (as well as the remaining ledges of the valve component 210), the upper portion 222 moves relative to the lower portion 224. In this manner, the protrusion 254 of the upper portion 222 enters the compartment 256. When the protrusion 254 contacts one or more surfaces that define the compartment 256, the valve component 210 is in the closed position and fluid is prevented from entering the channel 258. The ledges, including the number, the size, the shape and the material makeup thereof, and the area of the surface 226 can define a threshold pressure that maintains separation (i.e., no contact) between the protrusion 254 and the surface(s) of the compartment 256. The transition of the valve component 210 from the open position to the closed position may occur when the external pressure provided by the fluid exceeds the threshold pressure, and a transition back to the open position may occur when the external pressure provided by the fluid is lowered to the threshold pressure. Alternatively, the transition of the valve component 210 from the open position to the closed position may occur when the external pressure provided by the fluid is at least at the threshold pressure, and a transition back to the open position may occur when the external pressure provided by the fluid lowers to a value that is less than the threshold pressure.

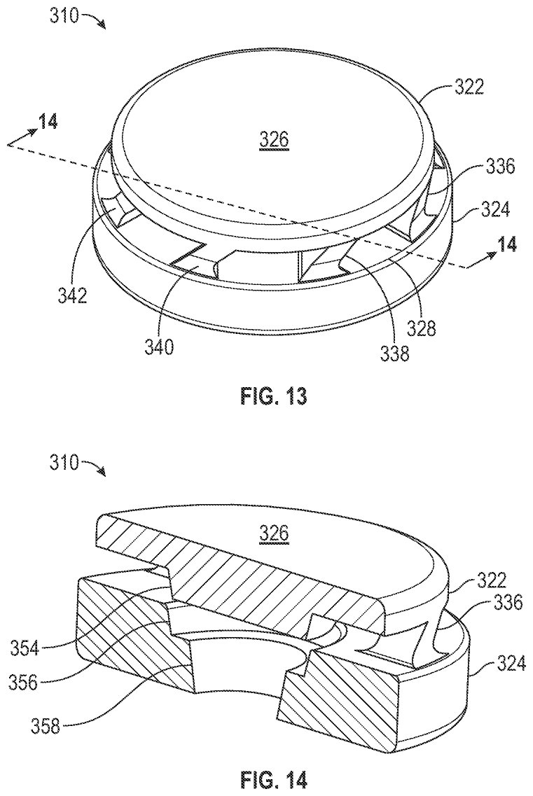

FIGS. 13 and 14 illustrate an alternate valve component 310, in accordance with aspects of the present disclosure. As shown, the valve component 310 includes an upper portion 322 and a lower portion 324. The upper portion 322 and the lower portion 324 may be referred to as a first portion and a second portion, respectively. However, “first” and “second” may be used interchangeably. Based on the respective positions, the upper portion 322 is positioned above the lower portion 324. The upper portion 322 and the lower portion 324 are separated by several ledges. For example, the valve component 310 includes a ledge 336, a ledge 338, a ledge 340, and a ledge 342. The ledges 336, 338, 340, and 342 may be representative of one or more additional ledges. Also, an opening is defined in part by adjacent ledges. For example, an opening 328 (representative of additional openings) is defined in part by ledges 336 and 338 (i.e., adjacent ledges).

FIGS. 13 and 14 show the valve component 310 is in an open position. As shown in FIG. 14, the upper portion 322 includes a protrusion 354 and the lower portion 324 includes a compartment 356 and a channel 358 fluidly connected to the compartment 356. In the open position, fluid flows between the upper portion 322 and the lower portion 324, into openings (i.e., opening 328 in FIG. 13) between adjacent ledges, through the compartment 356, and then exits the valve component 310 through the channel 358. However, the valve component 310 can transition from the open position to a closed position when a fluid applies sufficient pressure to a surface 326 of the upper portion 322. When the applied pressure by the fluid is greater than a threshold pressure provided the ledges 336 and 342 (as well as the remaining ledges of the valve component 310), the upper portion 322 moves relative to the lower portion 324. In this manner, the protrusion 354 of the upper portion 322 enters the compartment 356. When the protrusion 354 contacts one or more surfaces that define the compartment 356, the valve component 310 is in the closed position and fluid is prevented from entering the channel 358. The ledges, including the number, the size, the shape and the material makeup thereof, and the area of the surface 326 can define a threshold pressure that maintains separation (i.e., no contact) between the protrusion 354 and the surface(s) of the compartment 356. The transition of the valve component 310 from the open position to the closed position may occur when the external pressure provided by the fluid exceeds the threshold pressure, and a transition back to the open position may occur when the external pressure provided by the fluid is lowered to the threshold pressure. Alternatively, the transition of the valve component 310 from the open position to the closed position may occur when the external pressure provided by the fluid is at least at the threshold pressure, and a transition back to the open position may occur when the external pressure provided by the fluid lowers to a value that is less than the threshold pressure.

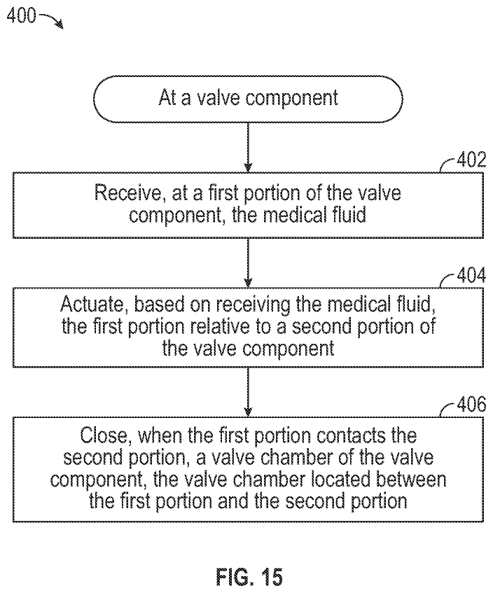

FIG. 15 illustrates a flowchart 400 showing a method for regulating a medical fluid through a fluid connector assembly, in accordance with aspects of the present disclosure. Valve components shown or described herein are capable of carrying out the steps of the method shown in the flowchart 400.

In step 402, the medical fluid is received at a first portion of the valve component. The medical fluid may be received through a fluid inlet of the fluid connector assembly. The medical fluid may include a saline solution, blood, medicaments, and/or total parenteral nutrition, as non-limiting examples. The first portion of the valve component may include an inner portion of the valve component. Alternatively, the first portion may include an upper portion.

In step 404, the first portion of the valve component is actuated, based on receiving the medical fluid, relative to a second portion of the valve component. The second portion may include an outer portion that surrounds the inner portion. Alternatively, the second portion may include a lower portion.

In step 406, a valve chamber of the valve component is closed when the first portion contacts the second portion. The valve chamber is located between the first portion and the second portion. The valve chamber, when closed, prevents the medical fluid from passing through the fluid connector assembly. The valve chamber represents a space or void between the first portion and the second portion. Initially, the first portion and the second portion are separated from each other, thus defining an opening position of the valve component. In some embodiments, the valve component transitions from the open position to the closed position when the fluid provides an external pressure that is at least at the threshold pressure, and the valve component transitions back to the open position when the external pressure is less than the threshold pressure. Alternatively, in some embodiments, the valve component transitions from the open position to the closed position when the fluid provides an external pressure that is greater than the threshold pressure, and the valve component transitions back to the open position when the external pressure is at least at the threshold pressure. Also, pressure is directly proportional to force, and as a result, a comparison between the external pressure and the threshold pressure is akin to a comparison between an external force and a threshold force, respectively.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 8 or clause 15. The other clauses can be presented in a similar manner.

Clause 1. A fluid connector assembly for a medical fluid, comprising: a housing comprising an internal chamber that forms a fluid path for the medical fluid; and a valve component disposed in the internal chamber, the valve component comprising: an inner portion, an outer portion, and a valve chamber between the outer portion and the inner portion, wherein the inner portion is movable between i) a first position, wherein the inner portion is separated from the outer portion, and ii) a second position, wherein the inner portion contacts the outer portion to seal the valve chamber and close the fluid path.

Clause 2. The fluid connector assembly of Clause 1, wherein the valve component comprises: a first surface comprising a plurality of first surface openings; and a second surface opposite the first surface, the second surface comprising a second surface opening fluidly connected to the plurality of first surface openings when the inner portion is in the first position.

Clause 3. The fluid connector assembly of Clause 2, wherein an external force provided by the medical fluid to the first surface causes the inner portion to move relative to the outer portion.

Clause 4. The fluid connector assembly of Clause 3, wherein: the inner portion is separated from the outer portion by the valve chamber, and responsive to the external force exceeding the threshold force, the inner portion is in the second position.

Clause 5. The fluid connector assembly of any of Clauses 2 and 3, wherein the second surface opening is fluidly connected to the plurality of first surface openings.

Clause 6. The fluid connector assembly of any of Clauses 1 to 5, wherein the valve component comprises a cylindrical valve component.

Clause 7. The fluid connector assembly of any of Clauses 1 to 6, further comprising a ledge that connects the inner portion with the outer portion.

Clause 8. A fluid connector assembly for a medical fluid, comprising: a housing comprising a housing chamber; and a valve component disposed in the housing chamber, the valve component comprising: a first portion, a second portion, and a valve chamber disposed between the first portion and the second portion, wherein responsive to receiving the medical fluid, the first portion moves relative to the second portion and closes the valve chamber.

Clause 9. The fluid connector assembly of Clause 8, wherein the second portion surrounds the first portion.

Clause 10. The fluid connector assembly of Clause 8, wherein, prior to an external force provided by the medical fluid, the first portion is separated from the second portion by a threshold force, and when the external force is at least at the threshold force, the first portion contacts the second portion.

Clause 11. The fluid connector assembly of Clause 10, wherein the valve component comprises: a first surface comprising a plurality of first surface openings; and a second surface opposite the first surface, the second surface comprising a second surface opening fluidly connected to the plurality of first surface openings by the valve chamber prior to the external force being at least at the threshold force.

Clause 12. The fluid connector assembly of Clause 11, wherein the first portion moves relative to the second portion based on the first surface receiving the medical fluid.

Clause 13. The fluid connector assembly of any of Clauses 8 to 12, further comprising a plurality of ledges, wherein the first portion is suspended from the second portion by the plurality of ledges.

Clause 14. The fluid connector assembly of any of Clauses 8 to 13, wherein the second portion remains stationary when the first portion moves.

Clause 15. A method for regulating a medical fluid through a fluid connector assembly, the method comprising, at a valve component located in the fluid connector assembly: receiving, at a first portion of the valve component, the medical fluid; actuating, based on receiving the medical fluid, the first portion relative to a second portion of the valve component; and closing, when the first portion contacts the second portion, a valve chamber of the valve component, the valve chamber located between the first portion and the second portion, wherein the valve chamber, when closed, prevents the medical fluid from passing through the fluid connector assembly.

Clause 16. The method of Clause 15, wherein: prior to an external force provided by the medical fluid, the first portion is separated from the second portion by a threshold force, and closing the valve chamber comprises receiving, by the medical fluid, an external force that is at least at the threshold force.

Clause 17. The method of Clause 16, further comprising opening the valve chamber when the external force is less than the threshold force.

Clause 18. The method of Clause 17, wherein opening the valve chambers comprises separating the first portion from the second portion.

Clause 19. The method of any of Clauses 15 to 18, wherein receiving the medical fluid at the first portion comprises receiving, at a first surface of the valve component, the medical fluid, the first comprising a plurality of first surface openings fluidly connected to the valve chamber.

Clause 20. The method of Clause 19, wherein closing the valve chamber comprises actuating the first portion toward a second surface of the valve component, the second surface comprising a second surface opening fluidly connected to the plurality of first surface openings.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A fluid connector assembly for a medical fluid, comprising:
a housing comprising an internal chamber that forms a fluid path for the medical fluid; and
a valve component disposed in the internal chamber, the valve component comprising:
an inner portion,
an outer portion, and
a valve chamber between the outer portion and the inner portion, wherein the inner portion is movable between i) a first position, wherein the inner portion is separated from the outer portion, and ii) a second position, wherein the inner portion contacts the outer portion to seal the valve chamber and close the fluid path.

2. The fluid connector assembly of claim 1, wherein the valve component comprises:
a first surface comprising a plurality of first surface openings; and
a second surface opposite the first surface, the second surface comprising a second surface opening fluidly connected to the plurality of first surface openings when the inner portion is in the first position.

3. The fluid connector assembly of claim 2, wherein an external force provided by the medical fluid to the first surface causes the inner portion to move relative to the outer portion.

4. The fluid connector assembly of claim 3, wherein:
the inner portion is separated from the outer portion by the valve chamber, and
responsive to the external force exceeding a threshold force, the inner portion is in the second position.

5. The fluid connector assembly of claim 2, wherein the inner portion has a first area defined by the first surface, and the inner portion has a second area defined by the second surface, and wherein the first area is greater than the second area.

6. The fluid connector assembly of claim 1, wherein the valve component comprises a cylindrical valve component.

7. The fluid connector assembly of claim 1, further comprising a ledge that connects the inner portion with the outer portion.

8. A fluid connector assembly for a medical fluid, comprising:
a housing comprising a housing chamber; and
a valve component disposed in the housing chamber, the valve component comprising:
a first portion,
a second portion, and
a valve chamber disposed between the first portion and the second portion, wherein responsive to receiving the medical fluid, the first portion moves relative to the second portion and closes the valve chamber.

9. The fluid connector assembly of claim 8, wherein the second portion surrounds the first portion.

10. The fluid connector assembly of claim 8, wherein,
prior to an external force provided by the medical fluid, the first portion is separated from the second portion by a threshold force, and
when the external force is at least at the threshold force, the first portion contacts the second portion.

11. The fluid connector assembly of claim 10, wherein the valve component comprises:
a first surface comprising a plurality of first surface openings; and
a second surface opposite the first surface, the second surface comprising a second surface opening fluidly connected to the plurality of first surface openings by the valve chamber prior to the external force being at least at the threshold force.

12. The fluid connector assembly of claim 11, wherein the first portion moves relative to the second portion based on the first surface receiving the medical fluid.

13. The fluid connector assembly of claim 8, further comprising a plurality of ledges, wherein the first portion is suspended from the second portion by the plurality of ledges.

14. The fluid connector assembly of claim 8, wherein the second portion remains stationary when the first portion moves.

15. A method for regulating a medical fluid through a fluid connector assembly, the method comprising, at a valve component located in the fluid connector assembly:
receiving, at a first portion of the valve component, the medical fluid;
actuating, based on receiving the medical fluid, the first portion relative to a second portion of the valve component; and
closing, when the first portion contacts the second portion, a valve chamber of the valve component, the valve chamber located between the first portion and the second portion, wherein the valve chamber, when closed, prevents the medical fluid from passing through the fluid connector assembly.

16. The method of claim 15, wherein:
prior to an external force provided by the medical fluid, the first portion is separated from the second portion by a threshold force, and
closing the valve chamber comprises receiving, by the medical fluid, an external force that is at least at the threshold force.

17. The method of claim 16, further comprising opening the valve chamber when the external force is less than the threshold force.

18. The method of claim 17, wherein opening the valve chambers comprises separating the first portion from the second portion.

19. The method of claim 15, wherein receiving the medical fluid at the first portion comprises receiving, at a first surface of the valve component, the medical fluid, the first surface comprising a plurality of first surface openings fluidly connected to the valve chamber.

20. The method of claim 19, wherein closing the valve chamber comprises actuating the first portion toward a second surface of the valve component, the second surface comprising a second surface opening fluidly connected to the plurality of first surface openings.
